(19) United States
(12) Patent Application Publication
(10) Pub. No.: US 2006/0129028 A1
(43) Pub. Date: Jun. 15, 2006

(54) Potency package

(76) Inventor: Alexander A. Krakousky, San Luis Obispo, CA (US)

Correspondence Address: JOHN R. ROSS, PO BOX 2138, DEL MAR, CA, 92014, US

(21) Appl. No.: 11/339,726

(22) Filed: Jan. 25, 2006

Related U.S. Application Data
(63) Continuation-in-part of application No. 10/813,714, filed on Mar. 31, 2004.

(51) Int. Cl. A61F 5/00

(52) U.S. Cl. 600/40; 600/38

(57) ABSTRACT

A device and method for male impotence correction and female anorgasmy. An electronic stimulator with at least one pulse generator is implanted inside the body. At least one electrode is installed in the epidural space in the sacrum section of the spinal column and a conductor running under the user's skin electrically connects the electrode to the pulse generator. The stimulator is programmable and may be controlled from outside the body. Upon command initiated by the user or the user's lover the stimulator produces very short low-voltage electrical pulses in the sacrum section that are picked up by the nerves leading to the sex organs of the user, which stimulates arousal in the user's reproductive systems. The pulses are similar to the pulses generated by heart pacemakers. In other preferred embodiments the stimulator includes one or two drug chambers and a tube extending from each chamber to a nerve for producing stimulation of a sex organ. The present invention works on both males and females. In a preferred embodiment, the programmable electronic stimulator is implanted under the skin in the patient's back. Stimulation of the nerves coming out from the parasympathetic part of the spinal cord causes dilatation of the penile arteries in the male and in the clitoris arteries of the female, which results in an erection in the male and pre-orgasmic sensation in the female. In female, the stimulation of the sacral part of the spinal cord increases sexual desire and escalation to the level of orgasm. A preferred embodiment provides for emission stimulation. Emission is stimulated by electrical excitation of the sacral part of the spinal cord by increasing the voltage of the previous impulses. The device may be preprogrammed to set in motion the emission and ejaculation process at a predetermined time interval after the start of the erection process.

The present invention is a continuation in part of Ser. No. 10/813,714, filed Mar. 31, 2004. This invention relates to medical devices and methods and particularly to devices and methods for the correction of impotence in males and anorgasmy in females.

### BACKGROUND OF THE INVENTION

### Sex Problems

Erectile function in male is dependent on the integration and regulation of functional interplay among psycologic, neurologic, endocrine and vasoactive factors. Parasympathetic division of autonomic nervous system located in the sacral region of the spinal cord primarily regulates human sexual function. Sexual performance in humans as well as in higher animals involves many functions. In males, there is erection, emission, ejaculation and orgasm. In females, there is initiation of sexual desire, escalation of the desire and orgasm. A wide variety of medical and psychological problems could interfere with one or more of these functions. The inability to achieve an erection is referred to as an erectile dysfunction or impotency. The inability to achieve an orgasm in females is referred to as anorgasmy. The principal methods presently used for male impotence correction and treatments include psychological and pharmacological treatments. Pharmacological treatments include noninvasive treatment (pills) and interventional treatment, which includes injection of vasoactive drugs into the penis. Surgical correction of impotence also exists. There is plastic surgery, prosthetic implantation and penile augmentation. There is no medical or surgical treatment that exists for anorgasmy in females at this time.

### The Spinal Column and the Spinal Cord

The central nervous system in humans is comprised of the brain and the spinal cord. Nerve fibers running within the spinal cord provide communication between the brain and various parts of the body. Some actions (reflex actions) are mediated through nerve connections in the spinal cord without involving the brain. Nerves carrying signals to the central nervous system are called afferent neurons and nerves carrying signals away from the central nervous system are called efferent neurons. The spinal cord is contained within the spinal column (also called the vertebral column). The spinal column is comprised of 26 irregular bones connected into a flexible curved structure. These are grouped into five sections. From the top down these five sections are the cervical curvature with 7 vertebrae, the thoracic curvature with 12 vertebrae, the lumbar curvature with 5 vertebrae, the sacrum with 5 fused vertebrae and the coccyx with 4 fused vertebrae. C, T, L, and S numbers (i.e., C1 through S5, numbered from the top of the cervical curvature to the bottom of the sacrum) identifies locations along the spinal column. See FIG. 17A. The spinal cord runs down from the brain through more than half of the spinal column. It terminates in or near the top of the lumbar curvature. Some nerves providing communication with the lower parts of the body continue on down through the spinal column. These nerves include the lumbar spinal nerves. The spinal cord and the lumbar spinal nerves are protected within the spinal column by a tough sheath called the spinal dural sheath. Just external to the spinal dura is an epidural space filled with fat and a network of veins. The fat forms a protective padding around the spinal cord. See FIG. 17B. The dura and the epidural space extend well beyond the end of the spinal cord. Nerves branch out from the spinal column throughout its length to serve the various sections of the body. For the most part separate sets of nerves are provided for the left and right sides of the body.

### Efferent Nerves

Sacral division of the parasympathetic part of autonomic nervous system is located on the level of S1 to S4. Pudendal nerve coming out from S2-S4 and becomes a major player in innervations of male and female genitalia. The pudendal nerve is an efferent nerve and is responsible for carrying much of the control information from the central nervous system to the genital region. This nerve identified as 17 in FIG. 1 and its main divisions are shown as dashed lines in the figure. The pudendal nerve has two divisions: perineal nerve and dorsal nerve of the penis or clitoris. After the pudendal nerve leaves pudendal canal it divides into the perineal nerve that travels alongside the ischiocavemous, bulbocavernous and bulbospondiosus muscle. During its pathway, fine nerve fibers course into the corpora spongiosum and scrotum. The dorsal nerve of the penis assembles into a network with the perineal nerves at the junction of corpus cavernosum and corpus spongiosum and joins the penile shaft just beneath the inferior pubic ligament. The main trunks of the dorsal nerve of the penis runs bilaterally at the base of the penis on 11 and 1 o'clock position, and lays on the surface of the tunica albuginea of the corpus cavernosum beneath the deep fascia of the penis (called the Buck's fascia) and innervates the penile shaft, urethra, glans of the penis and rectum. The superficial penile fascia (dartos fascia) and penile skin lay superficially over this layer. Final common pathway of the dorsal nerve of the penis represents by the cavernous nerves that innervate erectile tissue within the corpora cavernosa, seminal vesicles, prostate and urethra.

The pelvic nerve also known as an inferior hypogastric nerve comes out from lumbar (L4-5) and sacral division (S1-3). This nerve also innervates human genitalia and plays a big role in human sexual function.

### How It Works

FIGS. 5 and 6 show reproductive features of a male human. Erection of the penis is generally a necessary prerequisite for penetration of the vagina. The stimuli for this reflex may involve: (1) the sacral segment of the spinal cord where the pudendal nerve is initiated, (2) peripheral pudendal nerve receptor stimulation (around the penis) or (3) mental stimulation. The stimuli exist in the forms of electrical signals. These signals are transmitted via nerves. The afferent signals are transmitted via the pelvic nerves (right or left side) to the sacral segment of the spinal cord. Efferent signals are transmitted via the pudendal nerves. The pudendal nerves provide electrical signals to the penis arteries and a very large number of small arteries inside the penis in corpora cavernosa and corpora spongiosa. These electrical signals result in dilatation of the arteries permitting an increase of blood flow into the penis, which has the effect of partially restricting the veins taking blood out of the penis. As a result, there is a rapid filling of the blood spaces in the corpora cavernosum and corpus spongiosum areas of the penis. The swelling erectile bodies within the penis press on blood vessels draining the penis slowing the drainage. This physiologic effect makes the penis rigid. Therefore, the net effect is erection. Emission is the movement of spermatozoa and secretions from the testes and other accessory glands into the urethra. This is entirely a reflex process not involving the brain. The afferent side of the reflex arc is initiated by touch receptors in the genital area such as a receptor in the gland penis. Electrical signals travels via the pelvic plexus, the sacral segment of the spinal cord and the pudendal nerve to stimulate sympathetic fibers, which stimulate the ductus deferens to slow pump sperm and seminal fluid into the urethra. Ejaculation is the propulsion of the semen out of the urethra. The same afferent paths are involved. Central connection are located in the sacral segment of the spinal cord. Afferent and efferent stimuli are conveyed by pars sympathetic fibers of the pelvic splenic nerves and the pudendal nerves. Ejaculation is caused by the rhythmic contraction of the bulbocavernosus muscle, while the internal vesicle sphincter closes, preventing retrograde ejaculation into the bladder.

### Prior Art Patents

U.S. Pat. Nos. 5,246,015; 5,065,744 and 4,869,241 provide mechanical support for producing an erection. U.S. Pat. Nos. 5,236,904; 5,256,652 and 5,236,904 are the pharmaceutical type of impotence correction providing drugs administered to the penis. U.S. Pat. No. 5,454,840 issued to Applicant and one other describes a device and method for impotence correction. An electronic device is implanted inside the body. It is programmable and controllable from outside the body. The press of a button sends an electronic signal that initiates a process that simulates the body's natural reproductive processes. In a preferred embodiment, a programmable electronic device is implanted under abdominal muscle rectus. An electrical conductor is stitched to the surface of the pelvic splanchnic nerve. Stimulation of this nerve by a series of low voltage electrical pulses from the electronic device causes dilation of the penis arteries which results in an erection. The electronic device is controlled by a controller operated by the patient or his partner. Pat. No. '840 is incorporated herein by reference.

What is needed is an improved device and method for correcting dysfunctional impotence that simulates the natural processes of erection and ejaculation as closely as feasible.

### SUMMARY OF THE INVENTION

### Implantable Device for Stimulation of Nerves with Electrical Signals or Drugs

The present invention provides a device and method for male impotence correction and female anorgasmy. An implanted electronic device provides electrical pulses to stimulate nerves to produce desired sexual responses. These responses may include erection and emission in males and sexual excitement and organism in females. Nerves stimulated to produce these responses may include afferent nerves that transmit the stimulated signal to the spinal column. There the signal produces a response that is transmitted by efferent nerves to the genital region. Nerves can also be stimulated by electrodes located in the spinal column. The stimulated nerves can also include the efferent nerves that carry control information to the genital region. Preferred embodiments of the present invention provide preferred locations for the application of these electronic stimulations to these nerves. Other preferred embodiments describe techniques for the application of stimulating drugs at specific locations to provide relief from impotency problems. In other preferred embodiments closed loop techniques are described where sexual parameters are monitored with a sensor and processor that initiates the timing of the electronic stimulation or drug release based on the sensor information and a preprogrammed plan. Also, the present invention proposes various combinations of these techniques as needed to provide desired results.

### Direct and Indirect Stimulation

In U.S. Pat. No. 5,454,840 (hereinafter “the '840 patent) issued to Applicant and another, the inventors described a technique running conductors to an a nerve and connecting electrodes it where the nerve is clamped between two electrodes. Applicant refers to this and similar techniques as “direct nerve stimulation”. In the '714 parent application Applicant described a technique of inserting a needle type electrode in epidural space of the sacrum section of the spinal column. The electrode is not clamped to any nerve. Applicant refers to this and similar techniques as indirect stimulation. Direct stimulation can be applied to both afferent and efferent nerves. Indirect stimulation is normally applied at nerve endings or at the junction of nerves.

### Indirect Stimulation

In preferred embodiment of the present invention indirect stimulation is provided in the spinal column. The surgery in preferred embodiments involves only one or two electrode implanted the epidural space in the sacrum section of the spinal column and a conductor running under the user's skin electrically connects the electrode to the pulse generator. An electronic stimulator with at least one pulse generator is implanted inside the body. At least one electrode is installed in the epidural space in the sacrum section of the spinal column and a conductor running under the user's skin electrically connects the electrode to the pulse generator. The stimulator is programmable and may be controlled from outside the body. Upon command initiated by the user or the user's lover the stimulator produces very short low-voltage electrical pulses in the sacrum section that are picked up by the nerves leading to the sex organs of the user, which stimulates arousal in the user's reproductive systems. The pulses are similar to the pulses generated by heart pacemakers. The present invention works on both males and females. In a preferred embodiment, the programmable electronic stimulator is implanted under the skin in the patient's back. Stimulation of the nerves coming out from the parasympathetic part of the spinal cord causes dilatation of the penile arteries in the male and in the clitoris arteries of the female, which results in an erection in the male and pre-orgasmic sensation in the female. In female, the stimulation of the sacral part of the spinal cord increases sexual desire and escalation to the level of orgasm.

### Direct Stimulation

Other embodiments of the present invention provide for the electronic stimulation of efferent nerves that in normally carry control signals to the sex organs for example to produce erection or emission. Applicant discloses several preferred locations for applying electrical stimulation to these nerves to achieve desired results. Other embodiments provide for stimulation of afferent nerves that normally carry signals to the spinal column that in turn produce a response in one or more efferent nerves producing sexual responses.

### Closed Loop Embodiments

In other preferred embodiments sensors are utilized to monitor sexual events and a preprogrammed processor initiates the stimulation of nerves or drug injection to produce a sexual function such as erection, emission or orgasm.

### Combination of Techniques

Various combinations of the described above embodiments represent additional embodiments of the present invention.

### Female Anorgasmy

Female anorgasmy problems can be treated using substantially the same techniques as described in detail for men since the nerves serving the sex organs are substantially the same in both sexes.

### BRIEF DESCRIPTION OF DRAWINGS:

FIG. 1 is a drawings describing features of the human male reproductive system.

FIG. 2 is a drawing of the cross section of a human penis.

FIG. 3 is a drawing of a signal generator portion of a first preferred embodiment of the present invention.

FIG. 4 is the control unit of the device in FIG. 3.

FIG. 5 shows a preferred placement of the above first preferred embodiment in a human male.

FIG. 6 shows the function of the males reproductive system with the above preferred embodiment.

FIGS. 7A and B show a preferred method of connecting electrodes to nerve fibers.

FIG. 8 shows a signal generator portion of a second preferred embodiment.

FIG. 9 shows the function of the males reproductive system with the second preferred embodiment.

FIG. 10 shows a third signal generator, also incorporating a drug dispenser feature.

FIG. 11 shows where to connect the electrodes from signal generator shown in FIG. 7.

FIG. 12 shows a preferred pulse program for the FIG. 3 device.

FIG. 13 shows a preferred pulse program for the FIG. 8 device.

FIG. 14 shows stimulation in a section of the spinal cord.

FIGS. 15A and 15B show a signal generator for spinal cord stimulation and a control unit.

FIG. 16A and 16B show preferred pulse signals.

FIGS. 17A and 17B show features of a human spinal column and spinal cord.

FIG. 18 shows a pulse generator with a two-chamber pump.

FIG. 19 shows techniques for stimulating nerves in the spinal column.

FIGS. 20 and 21 show additional locations for stimulation.

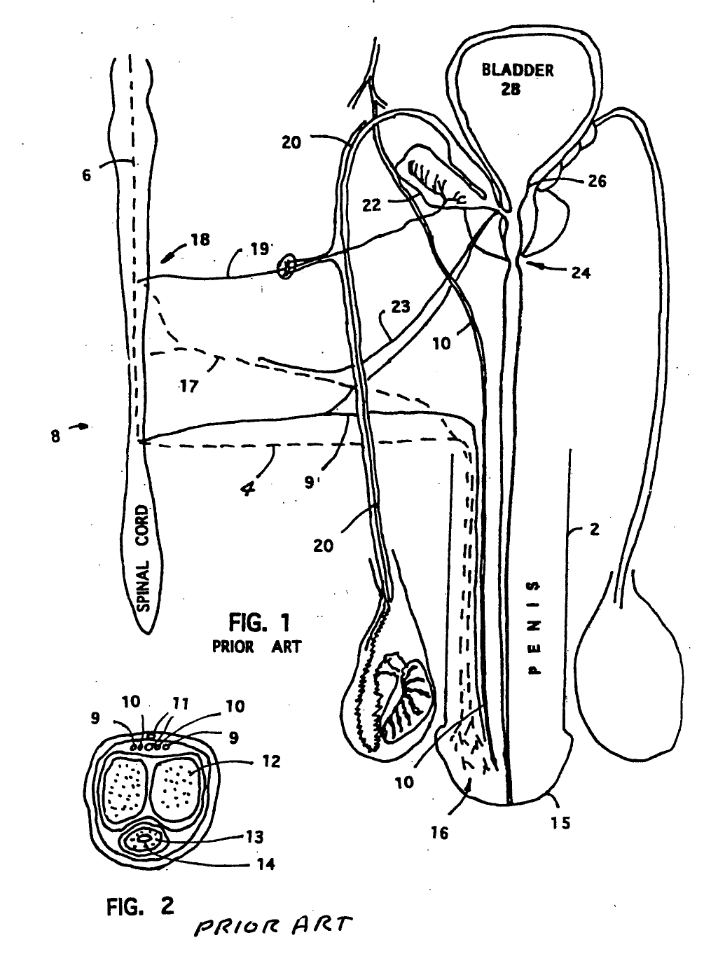

### DETAILED DESCRIPTION OF PREFERRED EMBODIMENTS

Preferred embodiments of the present invention can be described by reference to the figures.

### Exciting Afferent Nerves

Preferred techniques for producing errection in males is to utilize an electronic stimulator to excite afferent nerves (such as the pelvic splanchnic nerve) that carry information from the genital region to the spinal column. These nerve signals result in the stimulation of efferent nerves such as the pudendal nerve that in turn produce erection. As stated in the Background section of this application, the '840 issued to Applicant and another described techniques for dealing with impotency problems in males and females. This application is a continuation in part of Ser. No. 10/813,714, filed Mar. 31, 2004. Some of the new techniques described in this application are very similar to the techniques described in the '840 patent, so for completeness Applicant has included much of the description from both the '840 patent as well as the parent patent application.

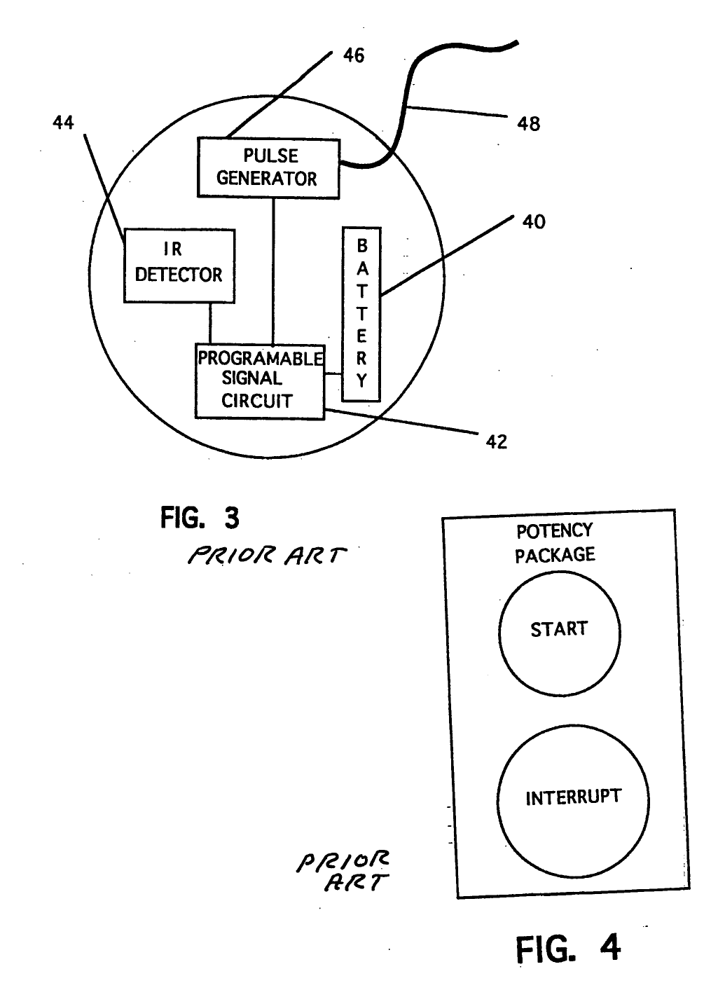

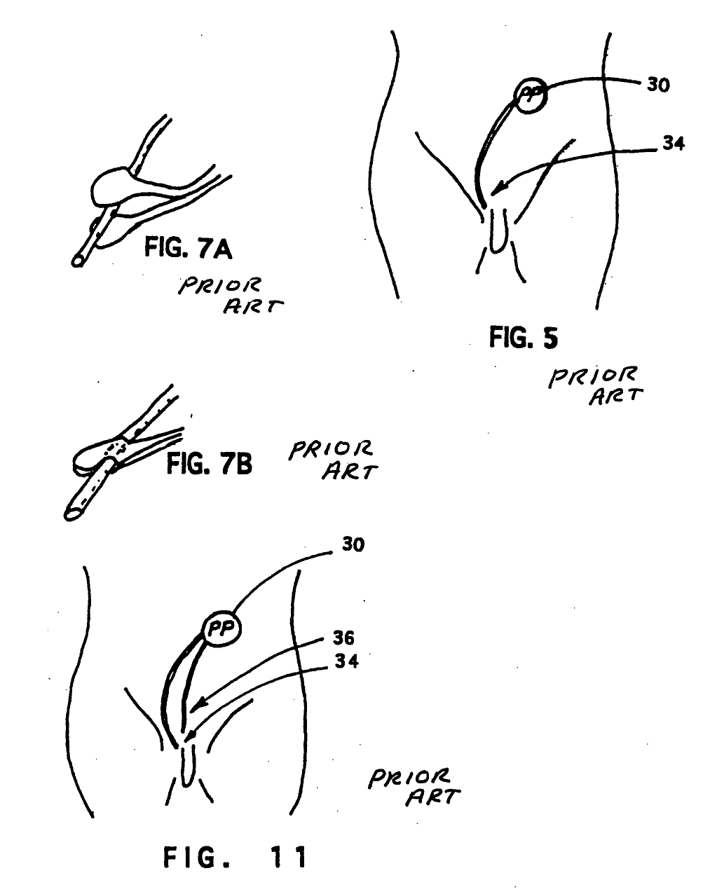

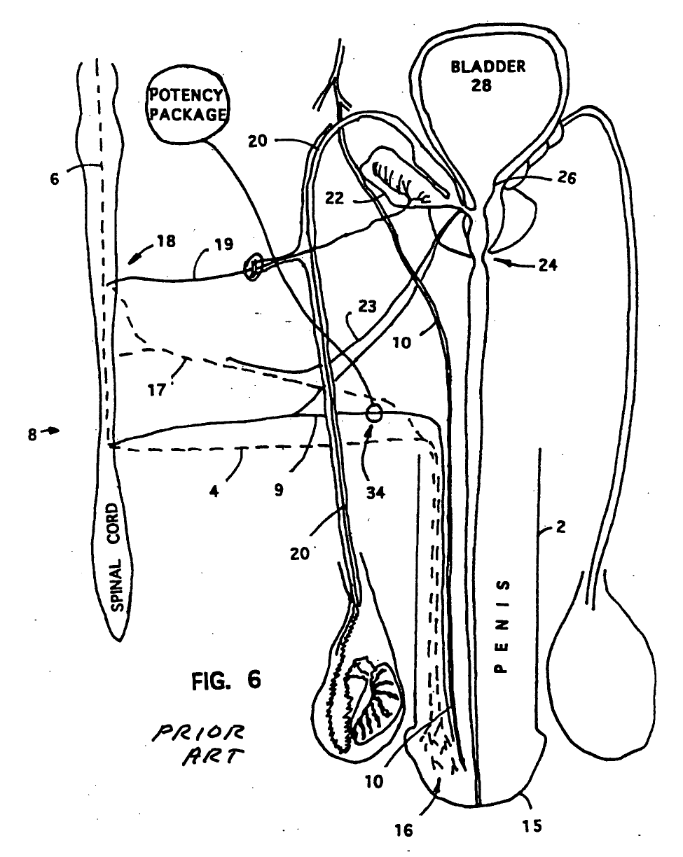

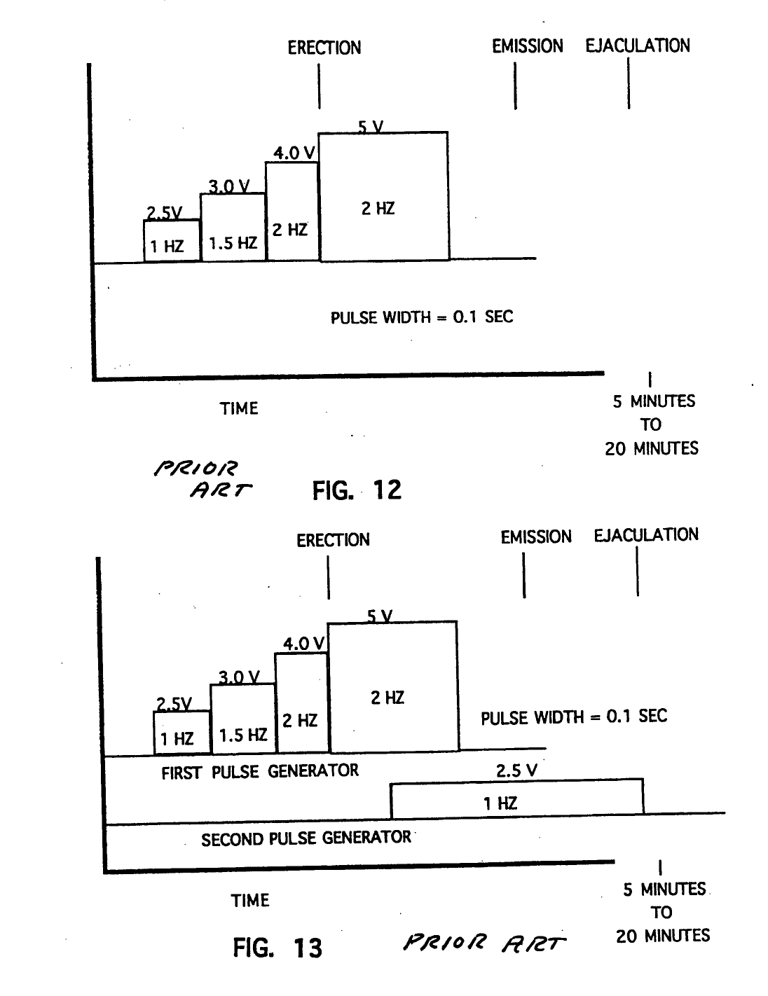

FIG. 3 is a copy of FIG. 3 of the 840 patent. It is a block diagram of a device called potency package by the Applicant and his co-inventor. A preferred prototype embodiment comprises a modified commercial pacemaker Model 600AV manufactured by Seimens. The device is designed to be installed under the patient's abdominal muscle rectus, but it could be installed in several other convenient places. The unit comprises battery 40, programable signal circuit 42, infrared detector 44 and pulse generator 46. The unit is controlled with an external control unit shown in block diagram for in FIG. 4. The unit comprises a start button and an interrupt button. A preferred sequence of pulses which should provide good results for many patients is shown in FIG. 12. The package can be reprogrammed to change any of the parameters shown in FIG. 12 which are pulse height, pulse width, frequency, duration and sequence. The best program for each individual patient can only be determined by testing. These parameters such as number of pulses group, voltage, pace, pulse duration are well within the ranges available with the above Seimens device. These parameters and any others within the range of the device can be programmed into it with commercially available pacemaker programmer such as Model #3CMHK 850 supplied by MIFI. The programmer transmits programming information via a pulsating magnetic field generated in the programmer to an electromagnetic detector in programmable signal circuit 42. This device shown in FIG. 3 comprises one electrode 48. The electrode should preferably be run from under the just patient's abdominal muscle rectus at position 30, under the abdominal skin tissue to location 34 as shown in FIG. 5 and there connect with the pelvic splanchnic nerves 9. The pelvic splanchnic nerve is located about one centimeter under the skin at the location shown on FIG. 5. The procedure can be accomplished in a medical doctors office or hospital, under local anesthesia. The nerve is located by a 5 cm incision at location 34. The nerve may be clamped between electrode leads as shown in FIGS. 7A and 7B. (Persons skilled in the art will recognize that many other surgical techniques for connecting electrodes to nerve tissue could be used.)

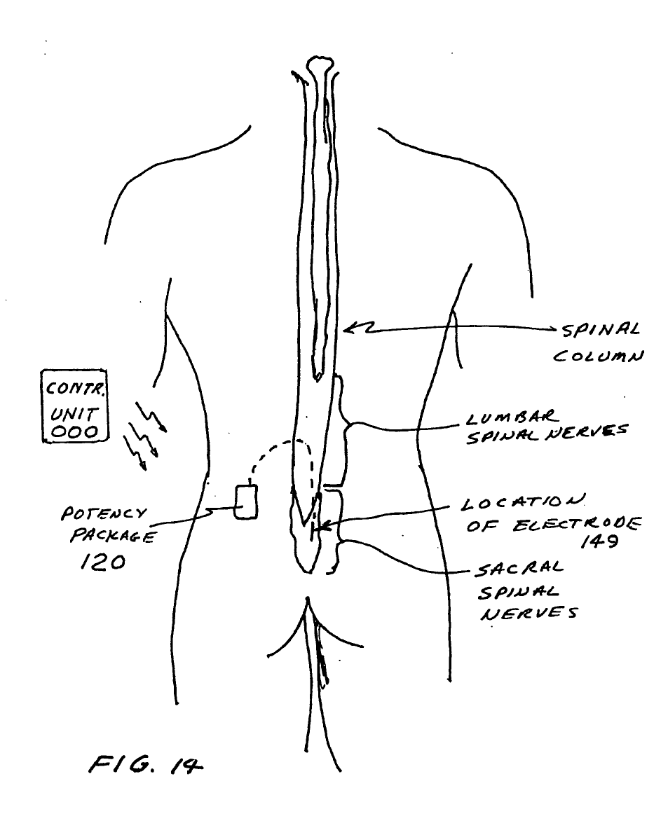

The recommended electronic pulse series is shown in FIGS. 12 and 13. If this series does not produce the desired effect, the doctor can vary the parameters. If an erection is produced by any of the tests, then the doctor continues the process and installs the potency package as described above. If the stimulator is unable to produce an erection, he may choose not to proceed with the operation.

### Equipment

The potency package components can be standard off-the-shelf components. The components include: a lithium battery LBSAR 5 made by SARATOF with a lifetime of 5 to 8 years, a pulse generator CLG 445 made by MIFI, a receiver/transmitter MC145027 made by Motorola and IR remote control receiver U338M made by AEG Corporation, a fast IR photodiode detector s1133-11 made by Hamamatsu, IR remote control transmitter U327M made by AEG Corporation, stepping motor 155 NL Micro Slide made by Toshiba Corporation, silicon tubing catheter T5715 made by Dow Coming Silasastic and elastomer Q7-4750 silicon pack made by Dow Corning Silastic.

### The Surgery for Direct Stimulation

The surgery to provide implantation is described with respect of the sixth embodiment which includes electrodes for erection and for emission and a thin tube for drug delivery to the penis. The potency package should be surgically implanted by a trained physician. The operation is very similar to the implantation of a heart pacemaker. A skin dissection is performed on the alba line below the umbilical. The peritoneal cavity is dissected and the path to the retroperitoneal is opened on the level of L4-S2. The electrodes are passed from the retroperitoneal to the subcutaneous layer of the frontal abdominal wall, where they are connected with the stimulator. Using micro surgical techniques, the carbonic electrode 28 is sewn to the parasimpatic nerve fiber and carbonic electrode 30 is sewn to the simpatic nerve fiber. The electrodes are passed from the retroperitoneal to the subcutaneous layer of the frontal abdominal wall where they are connected with the chambers 10 and 12 of the potency package. The tube carrying the drug is connected to the corpora cavernosa so as to deliver this drug to directly to the penis. The potency package is implanted subcutaneously to the frontal abdominal wall either to the right or to the left above the umbilical. The package has no contact with the operational wound. The wound is sewn layerly.

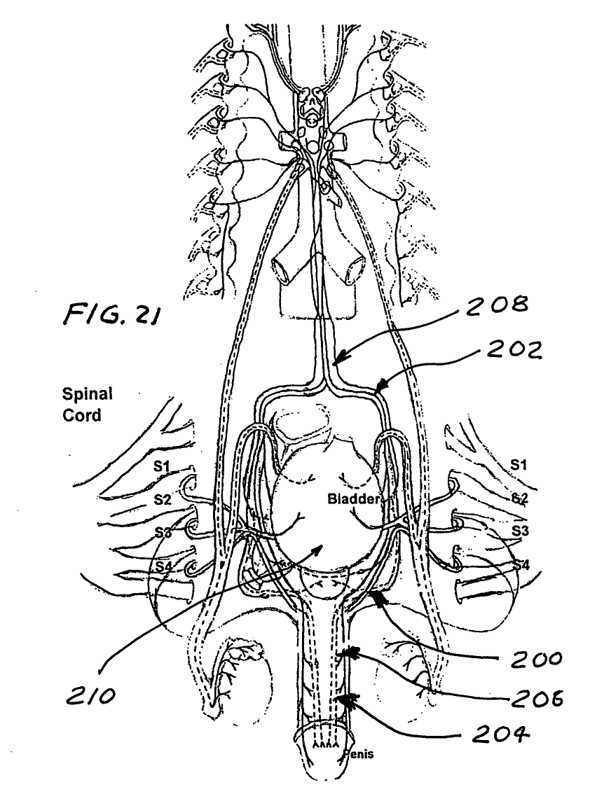

Stimulation of afferent nerves can be utilized to send signals to the spinal column where efferent nerves pick up the signal and transmit it out from the spinal column to produce a desired effect. For example direct electrode connections can be applied to the pelvic nerve and the hypogastric nerve, both of which are afferent nerves. Also, efferent nerves such as the pudental nerve. FIG. 21 shown a direct connection to the pudental nerve at 200. FIG. 21 also shows a direct connection to the deep nerve of the penis at 204. Direct stimulation of the cavernous nerves is shown at 206.

### Indirect Stimulation of Nerves in the Spinal Column

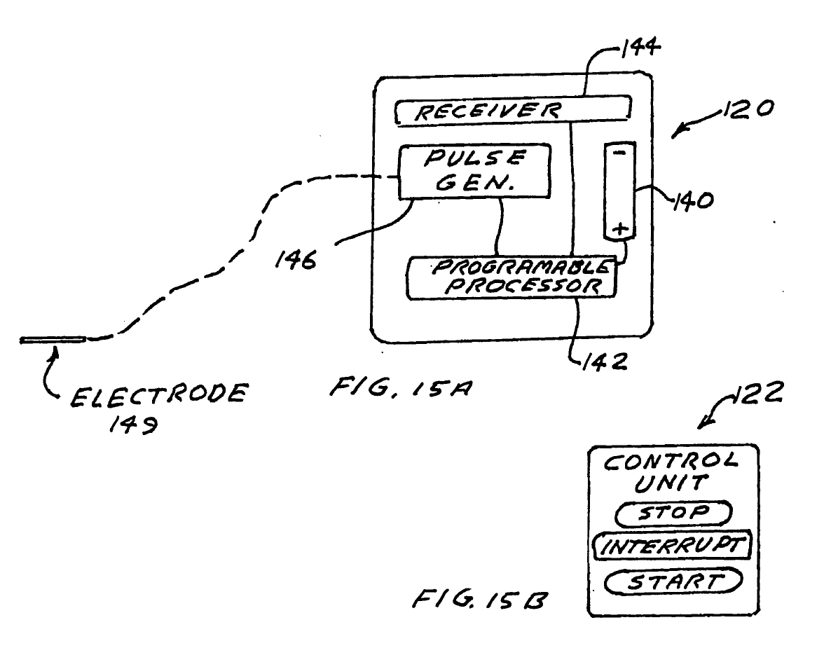

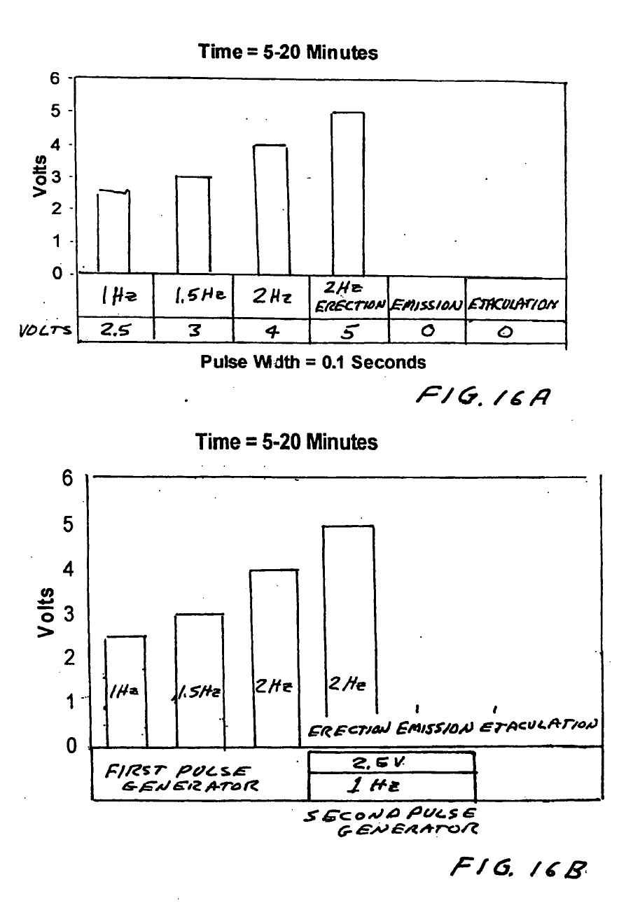

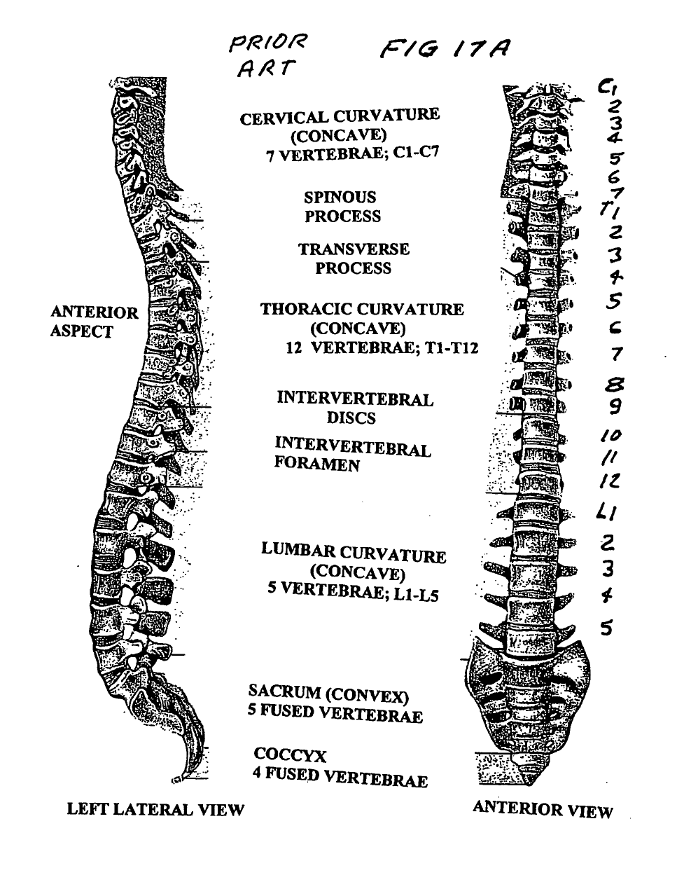

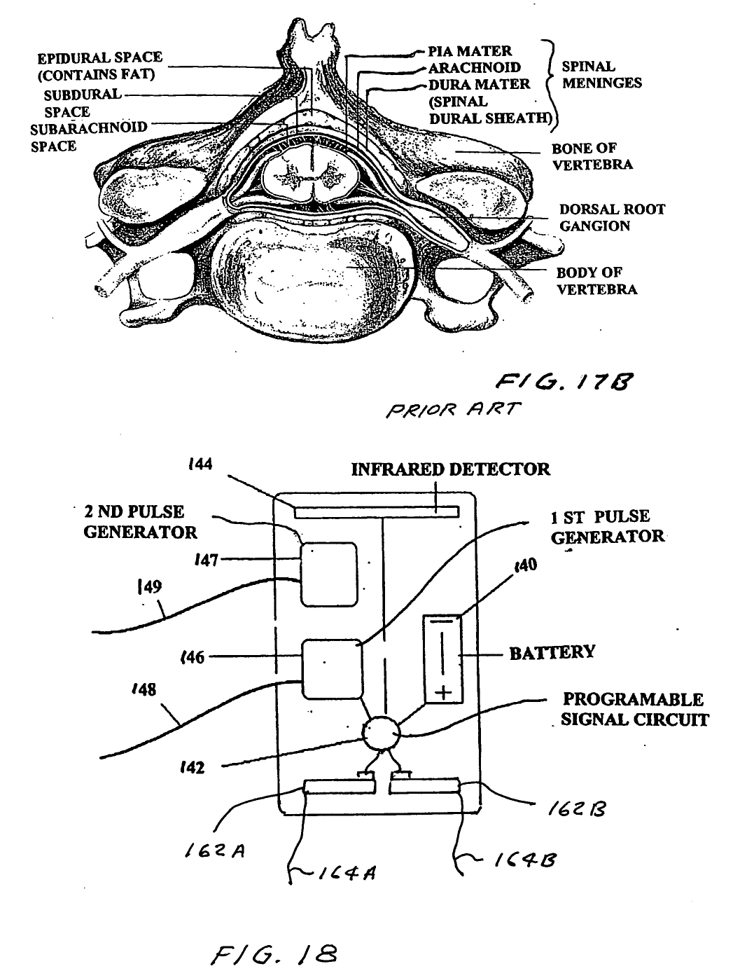

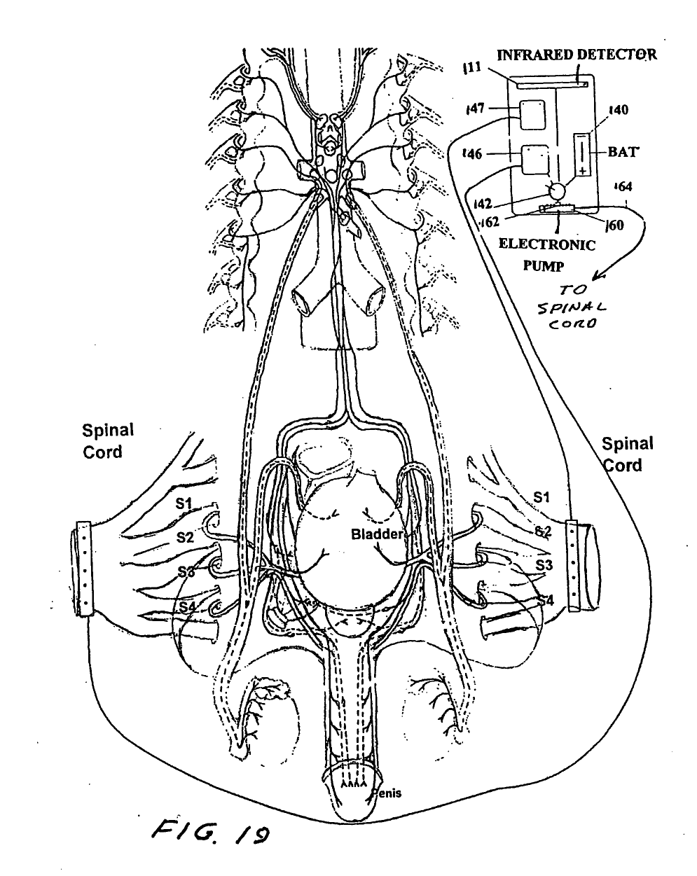

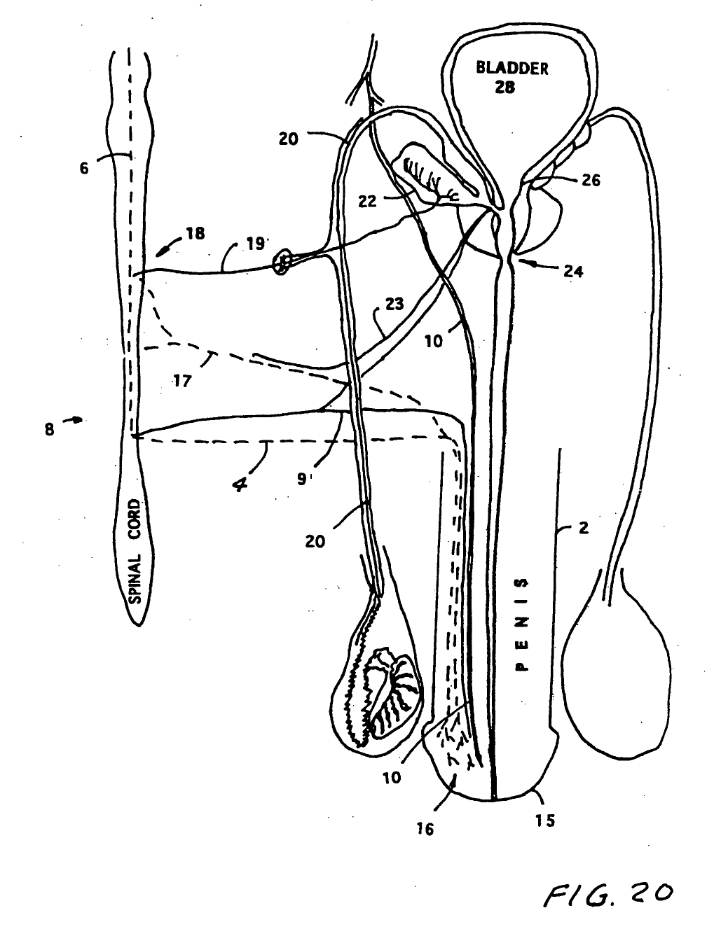

FIG. 14 shows a preferred embodiment of the present invention that was described in the parent of this application (Ser. No. 10/813,741). An two-inch long thin electrode 149 located in the epidural space at the S2, S3 and S4 level of the sacrum on the right side of the sacral spinal nerves is connected by an electrical conductor running under the skin of the user to a stimulator device 120 implanted under the skin of the user in the top part of the user's buttocks. FIG. 15A is a block diagram of the stimulator device, which the Applicant calls Potency Package Two. A preferred prototype embodiment comprises a modified commercial pacemaker Model 600AV manufactured by Siemens and modified by the Applicant. The unit comprises a battery 40, a programmable signal circuit 142, a pulse generator 146 and a receiver antenna 144. The electrode unit is a needle shaped with a lead that may be about 30 to 50 cm in length. The electrode element is positioned at the tip of the lead and typically has 4 or 8 ring type electrodes (each electrode having a length of 1 mm to 4 mm) with the electrodes spaced (edge to edge) about 1 mm to 4 mm apart with alternating polarity. These electrode units are available from Siemens and many other medical suppliers. These electrodes are not attached to any nerve but are typically located at the end of a single nerve or at the junction of two or more nerves. The unit is controlled with an external control unit 122 shown at FIG. 15B. The unit comprises a start button, stop button and an interrupt button. The preferred sequence of pulses that should provide good results for many patients, is shown in FIGS. 16A and 16B. The package can be reprogrammed to change any of the parameters shown in FIGS. 16A and 16B, which are pulse height, pulse width, frequency, duration and sequence. The best program for each individual patient can be only determined by testing. These parameters such as number of pulses grouped, voltage, rates and pulse duration are well within the range available with the above potency package device. These parameters are among others that could be programmed, with the range of the device, using a commercially available pacemaker programmer such as Model #3CMHK850 supplied by MIFI and SMHK and also described in Patent '840 referred to in the Background Section. The programmer transmits programmed information via a pulse site and magnetic field generated in the programmer, to the electromagnetic detector in a programmable signal circuit of the implanted device. This device, shown in FIG. 15A, comprises only one electrode. Before permanent implantation of the device, every patient has to go through the testing trial. The trial designed to detect the ability of the stimulation to achieve an erection. The approximately 2-inch long electrode runs from the epidural space at level S2, S3 and S4 of the sacral segment to under the patient's skin to connect the device. The procedure can be accomplished in any surgical center under monitored anesthesia care and local anesthesia. Personal surgical skill is required in order to install the device. The recommended electronic pulse series is shown in the FIG. 16A. If this series does not provide the desired effect, the doctor can vary the parameters. If an erection is produced by any of the tests, then the doctor continues the process and permanently installs the Potency Package Two as described above. If the doctor is unable to produce an erection in three days trial period, the doctor may choose not to proceed with the permanent implantation. In women patients, the clinical trial of the orgasm initiation stimulation contains the same approach in the same segment area in the sacral part of the spinal cord as in male patients. Instead of erection the doctor will be testing for pre-orgasm stimulation.

### Surgery for Indirect Stimulation

The details of the surgery designed to provide the implantation of the electrode and device as described above is now described. A trained surgeon should surgically implant the Potency Package Two device and electrodes. The operation is very similar to the implantation of the heart pacemaker. The patient should be previously anesthetized, spontaneously breathing with the application of standard monitoring by American Society of Anesthesiology, which includes EKG, blood pressure, pulse monitor and oxygen by nasal cannula. The patient should be put in the prone position on the operating table and the site on the lumbar part of the patient's spine should be prepped and draped in sterile fashion. Local anesthesia should be applied to the lower lumbar area, and a 2-cm incision of the skin in the middle of the spine should be performed. The epidural space should be identified using loss of resistance technique with a Tuohy needle and fluoroscopic imaging. When the epidural space on the left side is identified, the electrode should be transmitted through the needle, and the needle should be withdrawn. The electrodes should enter to the epidural space at the level of lumbar part of the spinal cord and advance down to the sacral region. The proper position of the electrode should be verified under fluoroscopy. The electrode should be advanced to the sacral segment of the spinal cord at the level of S2, S3 and S4 on the left side. If an electrode is to be applied to the right side instead of or in addition to the left side the same loss or resistance technique and fluoroscopic imaging has to be applied on the right side of the spine to identify the epidural space on the right side. The right-side electrode should be positioning on the same level on the right side of the patient's sacral segment, S2, S3 and S4. The proper position of the second electrode should be confirmed by the fluoroscopy. After the proper positioning of the electrode (or electrodes) on the side or sides of the patient's spinal nerves in the epidural space at the level of the sacral segment of the spinal cord, a voltage signal (at a low range of available voltages) with the proper setting of the impulses should be applied through the electrode to the spinal cord. The patient should be asked what kind of sensation in his or her genitalia has been felt. If the male patient has established the initiation of the erection process, the electrodes should be affixed at those levels. If the initiation of the orgasm in the female has been identified by electrical stimulation, the electrodes also have to affix and sutured at the level, which was identified.

The stimulator should be installed in the buttock area just below the waist on the left or on the right side as the patient prefers, a 5-cm incision should be made after application of local anesthesia to this area. The pocket for the pacemaker should be made there. The tunnel from the initial part where the electrode has entered to the patient's body at the level of the lumbar part of the spinal cord should be transmitted to the pocket area. By using a screwdriver, the electrode is connected to the stimulator, and the skin over the incisions should be closed.

### Stimulation of Efferent Nerves

Applicant is known world wide for his expertise in phalloplasty surgery. In the course of performing those surgeries Applicant has discovered that erection can be produced by the stimulation of Efferent nerves associated with the male sex organs. Since sexual stimulation of sex organs is virtually identical in males and females, Applicant is confident that sexual stimulation can also be achieved in females by the stimulation of their efferent nerves. The following are description of techniques for sexual stimulation by electrical stimulation of efferent nerves:

### Stimulation of the Pudendal Nerve and Its Divisions

Nerve signals carried by the pudendal nerve and its divisions cause a dilation of the penal arteries and a partial blockage of the penal veins. These effects produce erection. Applicant has discovered that direct and indirect stimulation of these nerves with low voltage pulses produce erections. The pudental nerve can be stimulated with indirect stimulation by positioning of a needle type electrode in the epidural space at the level S2-3-4 as shown at 149 in FIG. 14. FIGS. 16A and 16B show a recommended range of parameters for preferred electronic signals to be generated by the stimulator. Preferably, stimulation starts at relatively lower frequency and voltage and increases gradually as shown in the figures. However, each patient is an individual and for best results the pattern should be varied to determine the patterns that produce the best results for that patient.

### Indirect Stimulation of the Pelvic Nerve and the Hipogastric Nerve

For indirect stimulation of the pelvic nerve, the electrode should be positioned at the lumbar area of the spinal epidural space at the level L-2-3-4-5. These levels are indicated in FIG. 14 and 17A. The position is also shown at 202 In FIG. 21. Indirect stimulation of the hipogastric nerve can be done with the location of the electrode at position 208 as shown in FIG. 21.

### Stimulation to Produce Emission

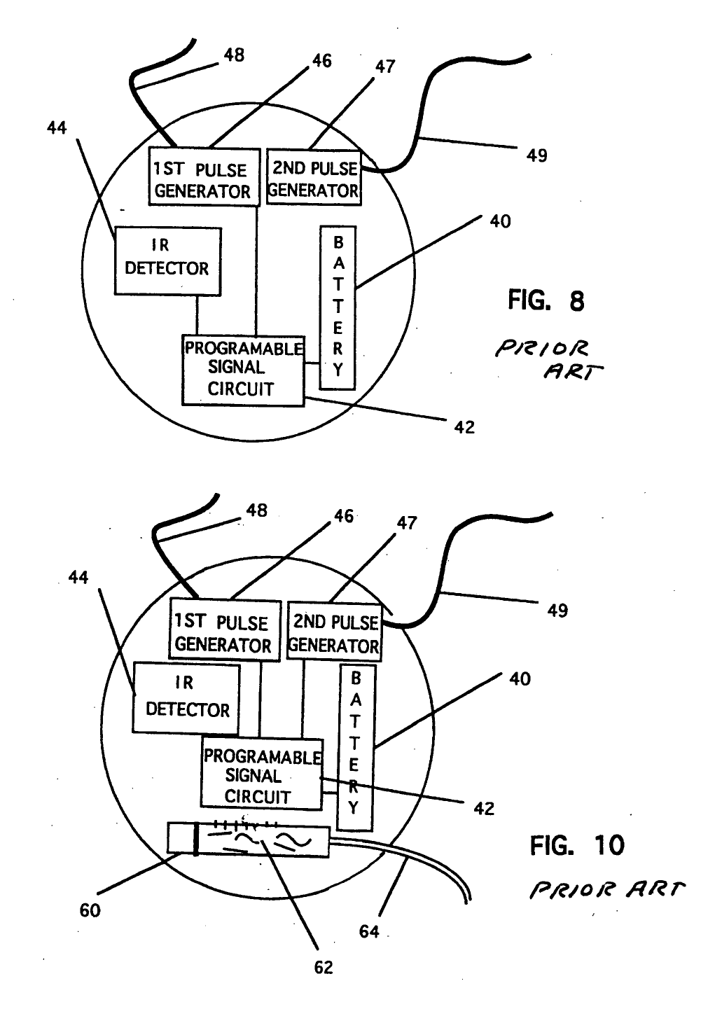

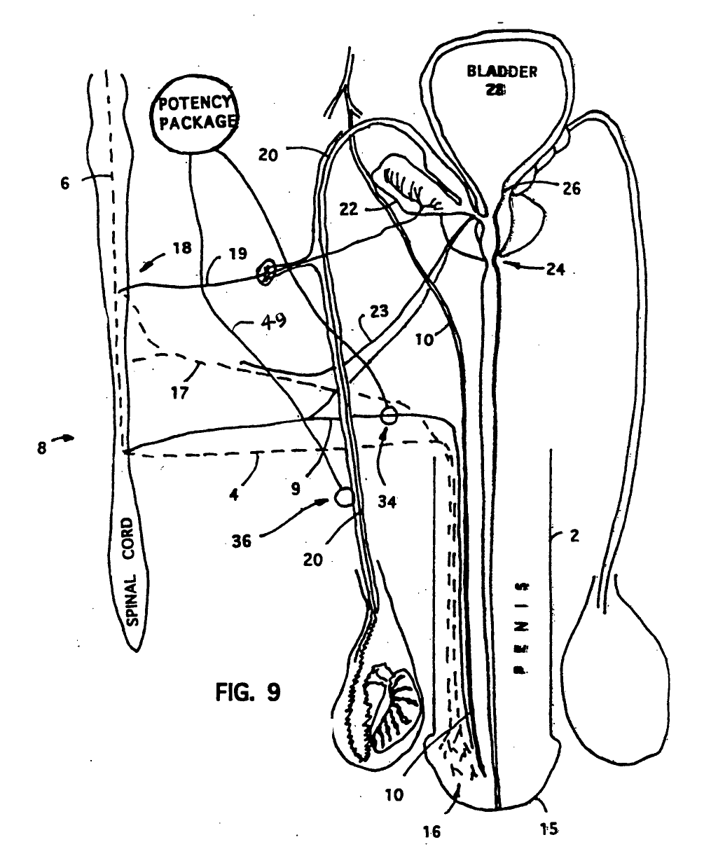

A second preferred embodiment described in the '840 patent is shown in FIG. 8. This embodiment is just like the first embodiment except the potency package comprises a second pulse generator 47 which is programmed to simulate emission. A second electrode 49 from the device is connected to the pelvic plexus nerve 19 at location 36 as shown in FIG. 9. Location 36 is also shown on FIG. 11. This nerve can most easily be reached by making a 1 cm incision of the skin in the pubic area just at the location shown at 36 in FIG. 11. Then make an incision of the rectal muscle ligament and reach the nerve-vessel-ductus deferens bundle. Make a 0.5 cm incision in the cover of the bundle and attach the electrode to the pelvic plexus nerve. The place of attachment is also shown in FIG. 9 at 36.

As above, if the doctor is uncertain if the patient is a good candidate for the procedure, Applicants recommend that a patient be tested prior to installation of the device to determine if the emission can be stimulated the particular patient. Applicants recommend that initially the potency package be programmed to produce the pulse trains shown in FIG. 13

### Stimulation with Drug Injection from Implant Device

In a preferred embodiment shown in FIG. 10, the stimulator contains a single-chamber 60 for storage within the body of a drug such papaverine and a small electronic pump 62 and a very thin tube for delivering of the drug to the spinal canal. The same result will be achieved with the delivering of the drug to the patient's body through the tube placed inside the spinal canal and deliver the drug in an on-and-off fashion to initiate the erection in the male. The delivery of the drug is initiated by an electronic signal transmitted by a hand-held transmitter controlled by the patient. For this alternative two electronic circuits are programmed as described above. The controller is programmed to deliver the drug at the time 0 which may be established using the hand held transmitter. A drug delivery single-chamber consists of plastic refillable containers, which is placed into hermetic chamber 62 as shown in FIG. 10. The bottom of the chamber is a piston with a coil and electromagnetic step driver 60. The first step of the erection stimulation is a vasoactive drug (such as papaverine) delivered by sending an electrical potential to the driver. The driver pulls the coil into the electromagnet to apply a force squeezing the drug into the tube attached to the plastic container at one end and implanted into the spinal canal at the other end of the tube. Chamber 62 is refillable upon injection through the skin with a syringe injector. These techniques can be applied to female patients. Tube 64 carries the drug to stimulate one or more nerves such as in the epidural space of the spinal column or in the penis.

### Dual Drug Chamber

In another preferred embodiment a stimulator is provided with a two-chamber 61 for storage of drugs as shown in FIG. 18. This two chamber device with chambers 162A and 162B permits the delivery of medications to two different places such as the spinal column and the penis through tubes 164A and 164B. This embodiment is also preferably programmable and operated using the hand-held transmitter as described above.

### Equipment

The potency package components can be standard off-the-shelf components. The components include: lithium battery, LBSAR5, made by SARATOF with a life-time of five to eight years, a pulse generator CLG445 made by MIFA, a receiver/transmitter MC145027 made by Motorola and IR remote control receiver 2338M made by AEG Corporation, and a fast IR Protodiode detector S113-11 made by Hamamatsu, IR remote control transmitter U327-M made by AEG Corporation, stepping motor 155ML microslide made Toshiba Corporation, Silicon tube catheter T5715 made by Dow Corning Silastic and elastimer Q74750 Silicon pack made by Dow Coming Silastic.

### Other Embodiments

Various combinations of the above-described embodiments provide many other embodiments of the present invention. The fourth embodiment would provide for emission stimulation only. A fifth is drug only. The following table lists vasoactive drugs and recommended quantities.

| | | |
|---|---|---|
| Papaverine | 15 | mg |
| Fentolamine | 0.5 | mg |
| Prostaglandin E1 | 20 | mcg |
| Vasoactive intestinal polypeptide | 5 | mcg |

In other embodiments two or more drugs may be administered using the implanted pump technique. For example, a drug such as nitroglycerin releasing into the blood stream to providing protection for patients against heart attack during sex. The following table shows some drugs recommended for correction of the most common health conditions occurring during sexual intercourse.

| | |
|---|---|
| Arrhythmia | beta-blockers |
| Asthma | alpha-blockers |
| Angina | nitroglycerin |
| Hypertension | beta-blockers |

Using many different drugs that are known to induce erection provides additional embodiments. Also, there are many electronic pulse sequences, which would work well to produce erection, emission and ejaculation for many different patients in addition to the sequences described above. Skilled doctors will recognize that electrodes can be connected at different locations other than those described above. To correct the arterial circulatory problem of impotence, the arterial anastomosis should be performed at the same time with the Potency Pack Two implantation. Also the penis enlargement surgery and the penis lengthening surgery could be applied at the same time as the implementation of the electrodes for the correction of male impotence. The vascular problems related to peripheral vascular disease should preferably be treated with anastomosis between inferior hypogastric arteries and the central and dorsal artery and dorsal vein of the penis.

Diabetic Type Impotency Correction

To correct the diabetic circulatory problem of impotence, the arterial anastomosis should be performed to the penis at the same time as the above procedures are implemented. Anastomosis between the hypo-gastric artery and dorsal artery should be performed end-to-end or end-to-side. The penile vein of diabetic patients can usually provide blood flow and surgical correction is not required in most cases.

Male Enhancement Surgery

The above technioques could be implanted during the period of penis enlargement by Dermograph, or by Allograft. They are implanted inside the penis with the preservation of the corpora cavernosum and the corpus spongiosum. The dermal graft or allograft is implanted under the skin of the penis with fixation to the distal part of the glans penis and the proximal part of the shaft of the penis. The penile lengthening is done by dissection of the tendon, which is affixed to the base of the penis and the pubic bone. Increase in length of the penis usually is from 1.0 inches to 1.5 inches. Increase in girth of the penis depends upon the patient's preference. The average increase in penis size is 20 to 30 percent.

Treatment of Anorgasmia in Females

The surgical technique described above for males can be applied to treat anorgasmy in women. The stimulation of the sacral part of the spinal cord generates impulses through the pudendal nerve that supplies innervation to the inner and outer part of the vagina and the clitoris. This artificial stimulation leads to the generation of the impulses that provide the additional blood supply and lubrication to the vagina and increase of the arousal part of the sexual desire in women. The additional stimulation technique applied through the nervous system allows women to feel a higher level of sexual arousal, increase escalation of the orgasm and the actual achievement of the orgasm. The higher level of sexual arousal increases the ability to achieve orgasm in the majority of human subjects.

Closed Loop Embodiments

In other preferred embodiments sensors are utilized to monitor sexual events and a preprogrammed processor initiates the stimulation of nerves or drug injection to produce a sexual function such as erection, emission or orgasm. Instead of using the hand-held stimulator to initiate the above described processes, the stimulator package could be preprogrammed to start the process based on a sensor signal. Here are some examples:
1) A timer could be includes to start the process at a particular time each night or twice per week such as 11:00 PM or at 11:00 PM on Wednesday and Saturday.
2) A voice recognition unit could be added to the package to start the process upon hearing the words, “I love you Darling”.
3) The package could include a pressure switch that starts the process when pressed.
4) The pressure switch could be located at a variety of places on the patient's body.
5) A heart-beat monitor could be included and the package could be programmed to start the process when the patient's heart beat increases by about 20 percent, such as from 70 bpm to 84 bpm.

Preferable in each of the above examples the package should include a technique for turning off the start process or canceling it if the process gets started unintentionally.

Animal Use

The present invention can be applied to many animals. It should be especially valuable for use with breeding animals such as prize bulls. It could also be used in a breeding program of captured members of endangered species in wild animals.

While the above description contains much specificity, the reader should not construe this as limitations of the scope of the invention, but merely as exemplification of preferred embodiments thereof. This skilled art will envision many other possible variations like another location for the stimulator, different types of electrodes and pacemakers, different voltages, amplitudes, pulse groups, repetition rate, pulse duration, remote control with more or less functions, fully automatic preprogrammed pacemaker without external controls, etc. Other elements in the woman's device might provide for an electrode for ureteral muscle contraction for urine incontinence correction during sexual intercourse. Additional electronic devices could be added to the potency package, such as a heart rate monitor a CO2 detector and a blood pressure detector. These devices could be designed to produce an alarm if the patient's data indicated that he or she is becoming too excited in the course of sex so that the patient and his partner can moderate their love making. Accordingly the reader is requested to determine the scope of the invention by the appended claims and their legal equivalence and not by the examples, which have been given.

What is claimed is:

1. A sex enhancement simulator for providing stimulation to a sex organ of a patient for correcting male impotence or female anorgasmy, said stimulator comprising:
1) a stimulator package installed under skin of a said patient, said stimulator package comprising:
a) at least one pulse generator,
b) a receiver for receiving instructions from said patient or said patient's lover,
c) a programmable processor for controlling said at least one pulse generator to produce desired pulse shapes upon receipt of said instructions,
d) at least one electrode implanted in the patient for direct or indirect stimulation of at least one nerve producing stimulation of a sex organ,
e) at least one electrical conductor located under said patient's skin and connecting said at least one electrode to said at least one pulse generator,
2) a controller for use by said patient or said patient's lover for transmitting instructions to said stimulator package.

2. The stimulator as in claim 1 wherein said electrode is located in an epidural space in said patient's sacrum for producing indirect stimulation of said at least one nerve.

3. The stimulator as in claim 1 wherein said electrode is attached to said at least one nerve producing direct stimulation of said at least one nerve.

4. The stimulator as in claim 3 wherein said nerve is an afferent nerve.

5. The stimulator as in claim 3 wherein said nerve is an efferent nerve.

6. The stimulator as in claim 1 wherein said stimulator also comprises at least one drug chamber and a tube leading from said chamber to at least one nerve producing stimulation of a sex organ.

7. The stimulator as in claim 1 wherein said stimulator also comprises at least two drug chambers and a tube leading from each of said two chambers to at least one nerve producing stimulation of a sex organ.

8. The stimulator as in claim 7 where one of the tubes leads to an epidural space of the spinal column and the other tube leads to the penis.

9. The device as in claim 1 wherein said at least one electrode is implanted in as epidural space at levels S2, S3 and S4.

10. A device as in claim 1 wherein said at least one pulse generator is two pulse generators and said at least one electrode is two electrodes and said at least one electrical conductor is two electrical conductors.

11. The device as in claim 1 wherein said patient is a male human.

12. The device as in claim 1 wherein said patient is a female human.

13. A device as in claim 1 wherein said stimulator package also comprises at least one storage vessel for the storage of vasoactive drug and said transmitting means for transmitting said vasoactive drug.

14. A device as in claim 1 wherein said stimulator package also comprises at least two storage vessels, each storage vessel adapted for the storage of vasoactive drug and said transmitting means for transmitting said vasoactive drug.

15. A device as in claim 1 wherein said stimulator package also comprises a storage vessel for the storage of vasoactive drug and said transmitting means for transmitting said vasoactive drug to said patient's sacrum.

16. A method of correcting male impotence correction and female anorgasmy in a patient comprising the steps of:
A) installing under the skin of the patient a stimulator package, said stimulator package comprising:
a) at least one pulse generator,
b) a receiver for receiving instructions from said patient or said patient's lover,
c) a programmable processor for controlling said at least one pulse generator to produce desired pulse shapes upon receipt of said instructions,
d) at least one electrode implanted in the patient for direct or indirect stimulation of at least one nerve producing stimulation of a sex organ,
e) at least one electrical conductor located under said patient's skin and connecting said at least one electrode to said at least one pulse generator,
2) controlling said stimulator package with a controller adapted to transmit instructions to said stimulator package.

17. The method as in claim 16 wherein said electrode is located in an epidural space in said patient's sacrum for producing indirect stimulation of said at least one nerve.

18. The method as in claim 16 wherein said electrode is attached to said at least one nerve producing direct stimulation of said at least one nerve.

19. The method as in claim 18 wherein said nerve is an afferent nerve.

20. The method as in claim 18 wherein said nerve is an efferent nerve.

21. The method as in claim 16 wherein said stimulator also comprises at least one drug chamber and a tube leading from said chamber to at least one nerve producing stimulation of a sex organ.

22. The stimulator as in claim 16 wherein said stimulator also comprises at least two drug chambers and a tube leading from each of said two chambers to at least one nerve producing stimulation of a sex organ.

23. The method as in claim 22 where one of the tubes leads to an epidural space of the spinal column and the other tube leads to the penis.

24. The stimulator as in claim 1 wherein the programmable processor is programmed to start a sexual stimulation process based on a signal detected by a sensor.

25. The stimulator as in claim 24 wherein said sensor is chosen from a group of sensors consisting of: a timer, a voice recognition device, a switch and a heartbeat monitor.
